(19) United States
(12) Patent Application Publication
(10) Pub. No.: US 2026/0260746 A1
(43) Pub. Date: Sep. 3, 2026

(54) MACHINE-LEARNING COMPUTER SYSTEMS AND METHODS FOR PREDICTING EFFICACY OF CHEMICAL AND BIOLOGICAL AGENTS FOR TREATING DISEASES, SUCH AS GASTROINTESTINAL CANCERS

(71) Applicant: Deep Rx Inc., Pittsburgh, PA (US)

(72) Inventors: Xinghua LU, Pittsburgh, PA (US); Lujia CHEN, Pittsburgh, PA (US); Min SUN, Pittsburgh, PA (US)

(73) Assignee: Deep Rx Inc., Pittsburgh, PA (US)

(21) Appl. No.: 18/879,529

(22) PCT Filed: Jun. 20, 2023
(86) PCT No.: PCT/US2023/068727

§ 371 Date: Dec. 27, 2024

Related U.S. Application Data
(60) Provisional application No. 63/355,725, filed on Jun. 27, 2022.

(51) Int. Cl. G16H 50/20; G06N 20/00

(52) U.S. Cl. CPC G16H 50/20; G06N 20/00

(57) ABSTRACT

Machine learning system employs causal discovery methods to identify genes that cause colorectal cancer when affected by genomic alterations. Co-expression patterns among their target differentially expressed genes (DEGs) are discovered to construct a set of “metagenes,” such that their expression values reflect the states of the cellular signaling system. Using the metagenes as features to represent tumors, a classification model is trained to predict whether the tumor cells of a patient are sensitive to chemotherapy and biological drugs.

### PRIORITY CLAIM

The present application claims priority to U.S. provisional patent application Ser. No. 63/355,725, filed Jun. 27, 2022, titled MACHINE-LEARNING COMPUTER SYSTEMS AND METHODS FOR PREDICTING EFFICACY OF CHEMICAL AND BIOLOGICAL AGENTS FOR TREATING DISEASES, SUCH AS GASTROINTESTINAL CANCERS.

### BACKGROUND

There are estimated 1.9 million incidence cases of colorectal cancer (CRC) worldwide in 2020. CRC is the second leading cause of cancer death, accounting for 9.4% of cancer deaths worldwide. At different stages of the disease course, over 60% of CRC patients receive chemical or biological agents in their treatments in one or more settings: pre-operational neoadjuvant therapy, post-operational adjuvant therapy, and palliative chemotherapy for metastatic patients.

Common chemical and biological agents for treating CRC, as well as other gastrointestinal (GI) cancers, include fluorouracil plus leucovorin (FULV), oxaliplatin, irinotecan, and bevacizumab (Bev). Different combinations of the above agents are used in clinical practice: FULV; FULV+oxaliplatin (FOLFOX); FULV+irinotecan (FOLFIRI); FOLFOX+Bev; and FOLFIRI+Bev. For neoadjuvant therapy, FOLFOX is the most used regimen; for adjuvant therapy, FOLFOX is the standard of care; for metastatic patients, FOLFOX+/−Bev and FOLFIRI+/−Bev are used.

Due to the high prevalence of adverse effects associated with each of the above agents, a patient treated with a chemo combination is highly likely to be treated with a drug that does not benefit the patient but results in significant adverse effects. In current clinical practice, there are no biomarkers that can predict the efficacy of the above agents, either as an individual or as a combination. Thus, precisely selecting an effective drug while avoiding non-beneficial agents is a critical problem in the care of cancer patients.

### SUMMARY

In one general aspect, the present invention is directed to computer systems and methods for training a model, through machine learning, to predict the efficacy of biological agents, such as regimens (like FOLFOX) or single drugs (like oxaliplatin or bevacizumab) in treating patients with a GI cancer, such as esophageal cancer, gastric cancer, and colorectal cancer. The machine learning model, referred to herein as the “COLOXIS” model, can also be used to predict the sensitivity of GI cancer patients to the regimes/drugs in which the regimens/drugs are commonly used. This can enable a clinician(s) to form individualized regimens by selecting effective drugs to treat GI cancer patients. These and other benefits that can be realized through embodiments of the present invention will be apparent from the description below.

### FIGURES

Various embodiments of the present invention are described herein by way of example in conjunction with the following figures.

FIG. 1 is a flow chart depicting a process for training a machine learning model for predicting the efficacy of chemical and biological agents for treating gastrointestinal cancer according to various embodiments of the present invention.

FIG. 2 is a graph showing how a model trained according to embodiments of the present invention can predict outcomes of CRC patients treated with FOLFOX. FIG. 2 shows Kaplan-Meir curves of predicted responders and non-responders of TCGA CRC patients treated with FOLFOX.

FIG. 3 includes graphs showing how the COLOXIS signature is associated with the prognosis of patients treated with FULV only. In FIG. 3, Kaplan-Meir curves of COLOXIS+ and COLOXIS− patients treated with FULV only in the C-07 trial.

FIG. 4A compares recurrence-free survival (RFS) between patients treated with FULV and FOLFOX in the group designated as COLOXIS+. Cox proportional hazard p-value and interaction p-value of the COLOXIS signature are shown. FIG. 4B compares RFS of patients treated with FULV versus FOLFOX in the group designated as COLOXIS−. FIG. 4C shows a multivariate Cox proportional hazard analysis in the group designated as COLOXIS+.

FIG. 5A compares RFS between patients treated with FLOX/FOLFOX (Bev=0) and FOLFOX+Bevacizumab (Bev=1) in the group designed as COLOXIS+. FIG. 5B shows a comparison of RFS between patients treated with FLOX/FOLFOX versus FOLFOX+Bev in the group designated as COLOXIS−. FIG. 5C shows a multivariate Cox proportional hazard analysis of the impact of Bev.

FIG. 6 is a process flow chart, according to various embodiments of the present invention, for predicting a drug response by a GI cancer patient using the machine learning model developed in FIG. 1.

FIG. 7 is a diagram of a computer system according to various embodiments of the present invention.

### DESCRIPTION

Common chemical and biological agents for treating colorectal cancer (CRC) and other gastrointestinal (GI) cancers include fluorouracil, leucovorin, oxaliplatin, and bevacizumab. Different combinations of these agents are widely used in treating CRC patients as distinct regimens, but there is no well-established biomarker or decision support system enabling clinicians to select among multiple candidate regimens that would be most effective for a given patient. This patent application describes an artificial intelligence system, the COLOXIS system or model, to support selecting effective drugs to form optimal regimens for treating CRC patients or other GI cancers, as the case may be. The machine learning system employs causal discovery methods to identify cancer driver genes and their target differentially expressed genes (DEGs) involved in the disease development process of CRCs (or other GI cancers, as the case may be). Co-expression patterns among these target DEGs are discovered to construct a set of “metagenes,” such that their expression values reflect the states of the cellular signaling system. Using the metagenes as features to represent tumors, a classification model (COLOXIS) is trained to predict whether tumor cells are sensitive to, for example, oxaliplatin and bevacizumab. A validation study using large-scale phase III clinical trial data has demonstrated that the COLOXIS model can predict patient response to individual drugs (oxaliplatin and bevacizumab) as well as a combination regimen, such as FOLFOX, which is a chemotherapy regimen made up of the drugs folinic acid (leucovorin, FOL), fluorouracil (5-FU, F), and oxaliplatin (Eloxatin, OX). Accurately predicting the efficacy of these drugs facilitates decision-making by clinicians and CRC patients.

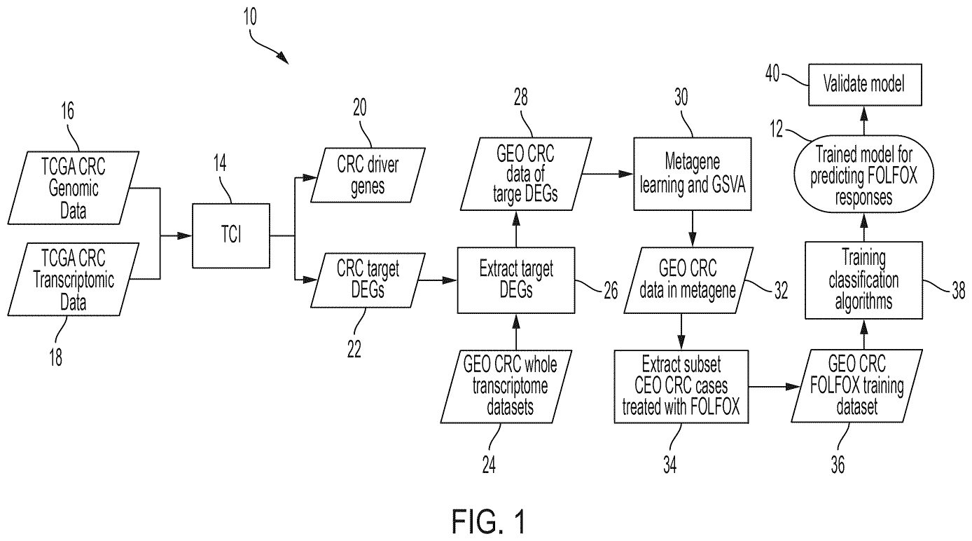

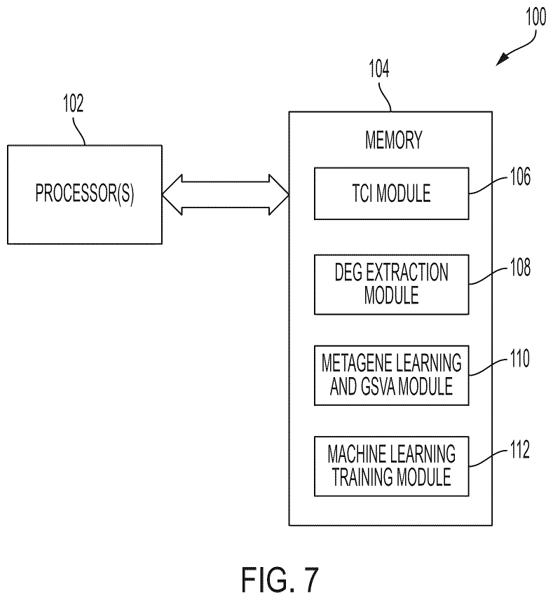

FIG. 1 is a flow chart depicting a process 10 for training a machine-learning model 12 for predicting the efficacy of chemical and biological agents for treating CRC according to various embodiments of the present invention. The development of the model (e.g., the aforementioned “COLOXIS model”) can consist of four major stages, which sequentially, in various embodiments, (I) model the disease mechanisms that influence heterogeneous response to drugs, (II) discover transcriptomic patterns reflecting disease mechanisms of cancer cells, (III) train models for predicting drug sensitivity based on cancer cell disease mechanisms, and finally (IV) validate the prediction models. Process 10 may be performed, in part or in whole, with a computer system, such as the computer system 100 described in connection with FIG. 7 below.

Stage I involves modeling heterogeneous disease mechanisms of CRCs using causal discovery methods. For each drug used to treat CRC, less than 40% of patients respond. Heterogeneous responses to drugs by cancer cells are due to the differences in disease mechanisms. That is, tumors have different disease mechanisms because of distinct driver somatic genome alterations (SGAs) in individual tumors perturbing signaling pathways, leading to different responses to a drug. Understanding the disease mechanisms of the cancer cells in a tumor would enable the prediction of drug responses of the tumor cells.

To investigate common disease mechanisms of CRCs, the tumor-specific causal inference (TCI) algorithm 14 can be applied to CRC genomic data 16 and CRC transcriptomic data 18. TCI is a Bayesian causal discovery algorithm invented by Drs. Xinghua Lu, Gregory Cooper, et al. from the University of Pittsburgh, described in U.S. Patent Application No. Ser. No. 16/349,192, published as Pub. No. 2019/0287651A1, which is incorporated herein by reference in its entirety. In various embodiments, 290 CRC tumors profiled by The Cancer Genome Atlas (TCGA) can be used as the CRC genomic and transcriptomic data 16, 18 to identify driver SGAs 20 and their target differentially expressed genes (DEGs) 22 in individual tumors. TCI searches for SGAs in a particular tumor that most likely cause a molecular phenotype (e.g., a DEG event) observed in the tumor. In experiments, the driver SGAs in individual tumors identified by the TCI algorithm were compiled. Of over 10,000 SGA-perturbed genes, 37 genes were designated major drivers of the CRC cohort. The discovery of drivers significantly narrowed down the number of candidate driver genes. Furthermore, the TCI analysis identified 2,691 genes that were regulated by these driver SGAs (i.e., target DEGs). Identification of driver SGAs 20 and their target DEGs 22 enables one to infer cancer cells' disease mechanisms (the states of signaling systems) based on genomic and transcriptomic data from a tumor.

Stage II involves discovering transcriptomic patterns reflective of the disease mechanisms of cancer cells. The expression status of a gene reflects the state of the signaling pathways regulating its expression, which can be used to infer the state of cellular signaling pathways. However, the expression values of an individual gene in different tumors are highly variable. Thus single-gene expression is an unreliable marker for inferring the state of signaling pathways. Since a signaling pathway usually regulates a set of genes (a gene module) in a cell, the expression status of a gene module is a better biomarker for inferring the state of a signaling pathway. Identifying the gene expression modules among the DEGs 22 discovered in Stage I would enable inference of the state of major signaling pathways perturbed in CRC, which can be further used for predicting drug responses.

In various embodiments, a database 24 of CRC transcriptome data is used to extract target DEGs at step 26. In various embodiments, the Gene Expression Omnibus (GEO) database with transcriptomic data may be used as the database 24. The inventors' experiments collected 4,199 CRC tumors from the GEO database. The expression values, at block 28, of the 2,691 target DEGs (see block 22) in these 4,199 CRC tumors are extracted at step 26. The extraction step 26 can involve, in various embodiments, a series of consensus clustering analyses to identify a reduced set of (e.g., ten to fifty, inclusive, a preferably around 15) co-expression modules (metagenes) that exhibited clean co-expression patterns and provided strong signals with respect to drug responses. Consensus clustering is a method of aggregating (potentially conflicting) results from multiple clustering algorithms. It refers to the situation in which a number of different (input) clusterings have been obtained for a particular dataset, and it is desired to find a single (consensus) clustering, e.g., the target DEGs, which is a better fit in some sense than the existing clusterings. An example of a suitable extraction technique is described in Monti S., Tamyo P, Mesirov J, Golub T., “Consensus Clustering: A Resampling-Based Method for Class Discovery and Visualization of Gene Expression Microarray Data,” Machine Learning 2003; 52:91-118, which is incorporated herein by reference. The Monti consensus-clustering algorithm described in this paper is used to determine the number of clusters, K. Given a dataset of N total number of points to cluster, this algorithm works by resampling and clustering the data, for each K and a N×N consensus matrix is calculated, where each element represents the fraction of times two samples clustered together. A perfectly stable matrix would consist entirely of zeros and ones, representing all sample pairs always clustering together or not together over all resampling iterations. The relative stability of the consensus matrices can be used to infer the optimal K.

Expression values of metagenes in each tumor can be estimated at step 30 using the gene set variation analysis (GSVA) method. Expression value (block 32) can then be used as features to represent the cellular states of cells in tumors. GSVA calculates sample-wise gene set enrichment scores as a function of genes inside and outside the gene set, analogously to a competitive gene set test. Further, it estimates variation of gene set enrichment over the samples independently of any class label. Conceptually, this methodology can be understood as a change in coordinate systems for gene expression data, from genes to gene sets. This transformation facilitates post-hoc construction of pathway-centric models, such as differential pathway activity identification or survival prediction. An example of a suitable GSVA method is described in Hanzelmann S, Castelo R, Guinney J., “GSVA: gene set variation analysis for microarray and RNA-seq data,” BMC Bioinformatics 2013; 14:7, which is incorporated herein by reference.

Stage III involves training classification models for predicting drug sensitivity. From a collection of datasets, such as GEO datasets (GSE19860, GSE28702, GSE72970, GSE69657, GSE104645), a cohort of mCRC patients can be identified at step 34 as a discovery dataset at block 36, with patients in the cohort having been treated with, for example, a FOLFOX regimen. The treatment responses were measured as RECIST (Response Evaluation Criteria in Solid Tumors) scores. In various embodiments, for each patient, a feature vector consisting of GSVA scores of a relatively small number of metagenes (e.g., 10-50 metagenes) can be constructed. In various embodiments, 15 metagenes can be used. Based on the RECIST scores, a class label can be assigned as a “responder” (CR and PR) or “non-responder” (SD or PD) to each patient in the training dataset. Based on this reduced dimension training data 36, the COLOXIS model 12 can be trained at step 38. In various embodiments, the COLOXIS model 12 is a regularized logistic regression model. The regularized logistic regression model (or other machine learning classifier as the case may be) can be trained at step 38 to predict response to FOLFOX in these mCRC patients. As explained further below, the performance of model 12 can be evaluated through cross-validation and external validation experiments. In other embodiments, other machine learning models could be trained besides a regularized logistic regression model. In other embodiments, the machine learning model 12 trained at step 38 from dataset 36 can be a deep learning artificial neural network, a support vector machine and/or a decision tree, or an ensemble of machine learning models, for example.

Stage IV involves validation, at step 40, of the prediction accuracy of the COLOXIS model 12. In various embodiments, the validation was in adjuvant therapies of CRCs. The clinical utility of the COLOXIS model 12 can be validated using data from the TCGA and from two Phase III clinical trials.

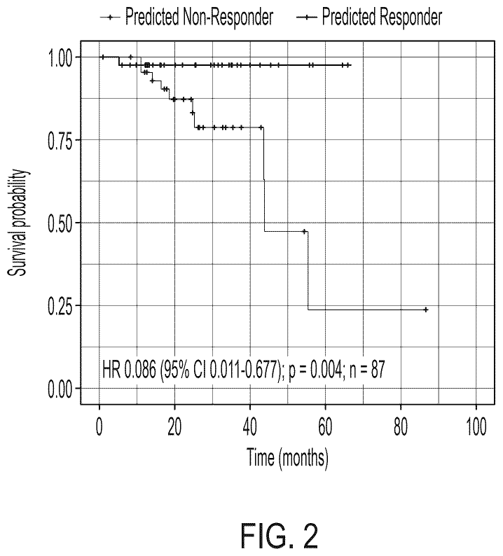

The inventors had the COLOXIS model 12 evaluated for predicting response to FOLFOX for CRC patients. From the TCGA study, genomic and clinical data were collected from 87 patients treated with the FOLFOX regimen and with known overall survival outcomes. The gene expression data of the patients were transformed using the GSVA algorithm to project patients in a 15-metagene space, and the COLOXIS model 12 was applied to predict whether a patient would respond to FOLFOX or not (referred to as COLOXIS+ and COLOXIS− groups). The survivals of the two groups of patients were compared. The results, as shown in FIG. 2, show that patients assigned to the COLOXIS+ group had significantly better overall survival.

The inventors also had the prognostic value of the COLOXIS signature evaluated. The COLOXIS model 12 was applied to a cohort of 1,285 colon cancer patients who had undergone post-surgery adjuvant therapy, studied in two Phase III clinical trials (the C-07 and C-08 trials), which were conducted by a non-profit clinical trial management organization, the National Surgery Adjuvant Breast and Bowel Project (NSABP). The C-07 trial compared the efficacies (in terms of preventing recurrence) of two chemo combinations: (1) fluorouracil plus leucovorin (FULV); and (2) FULV plus oxaliplatin (FOLFOX). The C-08 trial compared the efficacies (in terms of preventing recurrence) of two chemo combinations: (1) FOLFOX; and (2) FOLFOX+Bev.

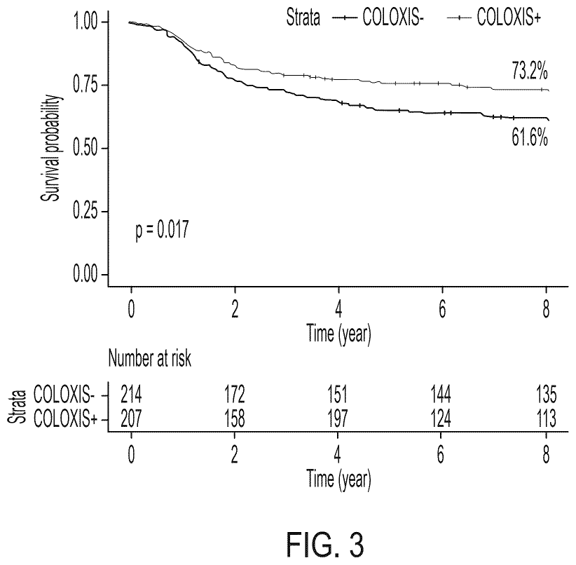

The prognosis value of the COLOXIS model 12 was evaluated by comparing the recurrence-free survival (RFS) of COLOXIS+ vs COLOXIS− groups among patients treated with FULV alone. As shown in FIG. 3, the COLOXIS signature is statistically significantly associated with the outcomes of patients (HR: 1.52, 95% CI=1.07 to 2.15, P=0.017).

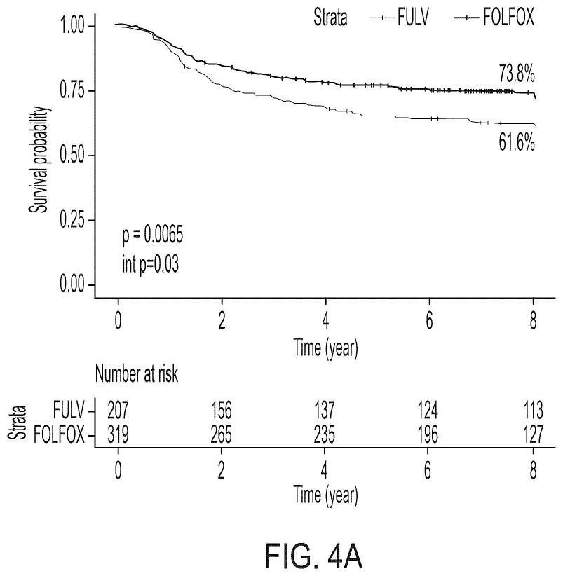

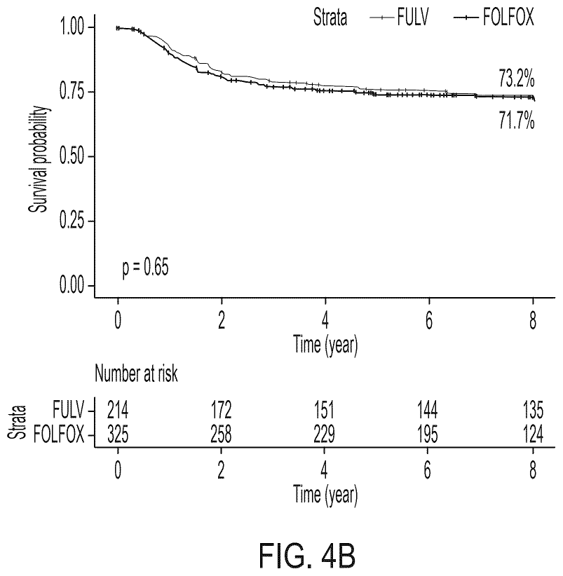

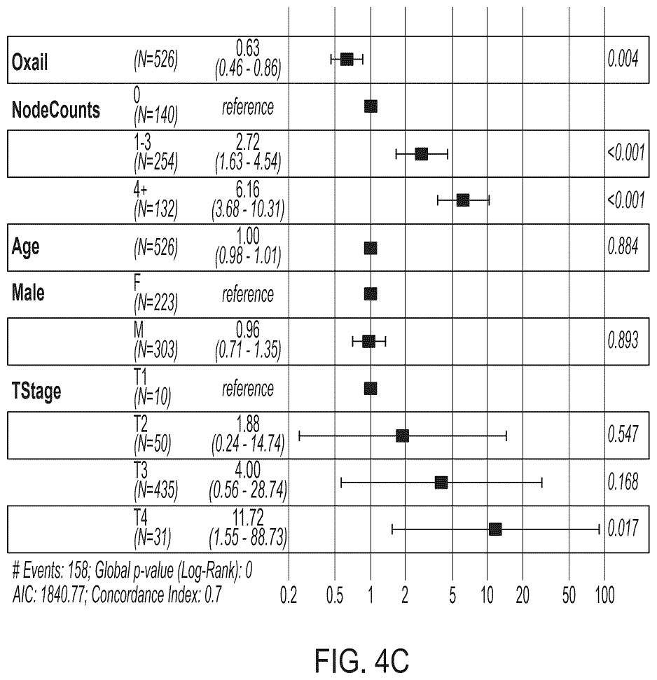

The inventors also evaluated the COLOXIS model in predicting oxaliplatin benefit. Among 1,065 patients treated with fluorouracil plus leucovorin (FULV) (N=421) and FOLFOX (N=644), 526 were predicted as benefiting from oxaliplatin-containing regimens (referred to as COLOXIS+) and 539 as not (COLOXIS−). The predictive value of the COLOXIS model was examined by comparing the response to oxaliplatin treatment in the COLOXIS+ vs. COLOXIS− groups. As shown in FIGS. 4A-C, the COLOXIS+ patients benefited from oxaliplatin (HR=0.65, 95% CI=0.48-0.89, P=0.0065, int P=0.03), but COLOXIS− did not (COLOXIS-HR=1.08, 95% CI=0.77-1.52, P=0.65). Thus, the COLOXIS signature can predict the oxaliplatin benefit.

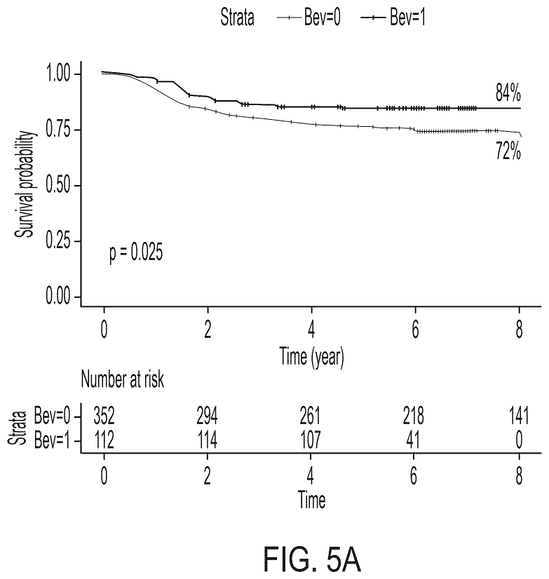

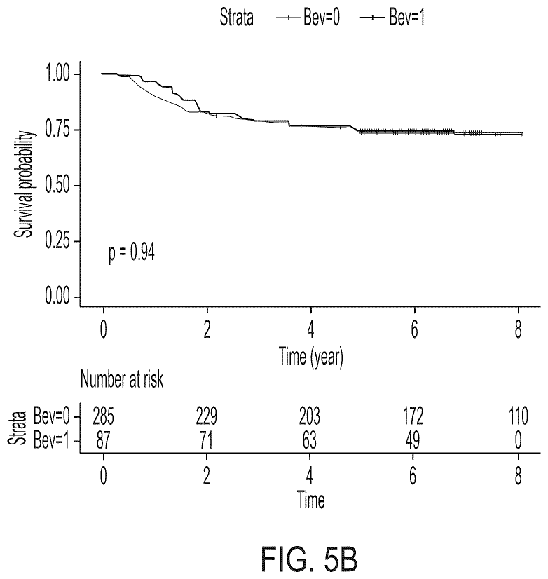

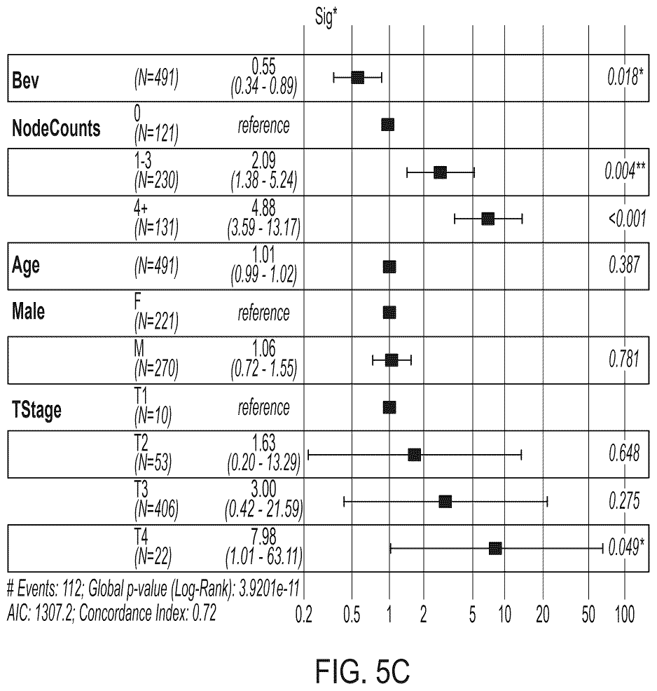

The inventors also evaluated the COLOXIS model in predicting the benefit of FOLFOX+Bev. Among 644 patients treated with FOLFOX and 219 patients treated with FOLFOX+Bev, 491 were assigned as COLOXIS+, and 372 were assigned to COLOXIS− group. The predictive value of the COLOXIS model was examined by comparing the response to adding bevacizumab in treatment in the COLOXIS+ vs COLOXIS− groups. As shown in FIG. 5C, the COLOXIS+ group significantly benefit from adding bevacizumab to FOLFOX (HR=0.58, 95% CI=0.36-0.94, p=0.025, int p=0.101), whereas COLOXIS− group did not (HR=1.02, 95% CI=0.64-1.63, p=0.94). Thus, the COLOXIS signature can predict the benefit of bevacizumab.

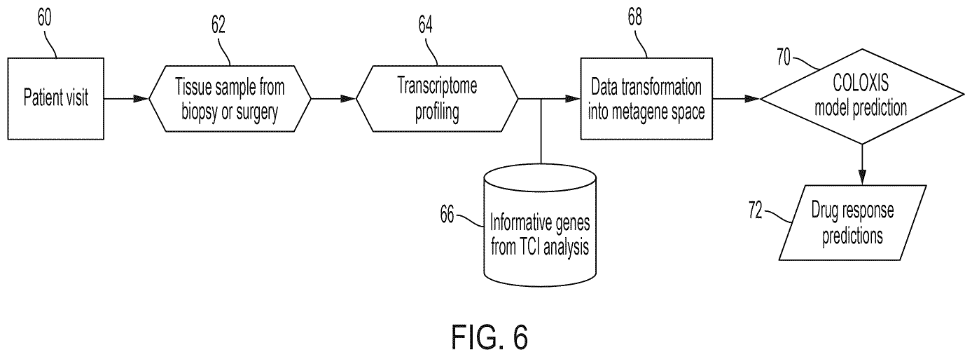

Once model 12 is trained and validated, it can be used as a diagnostic tool to predict how a patient will respond to an agent. For example, if trained as explained above, the COLOXIS model could be trained to predict whether a CRC patient will benefit from FOLFOX, oxaliplatin, or bevacizumab. FIG. 6 is a flow chart of a process for using the model as a decision support tool according to various embodiments of the present invention. At step 60, a patient visits a medical provider and is diagnosed with GI cancer, which gives rise to the need to make a decision regarding whether the patient's tumor cells are sensitive to oxaliplatin, bevacizumab or FOLFOX regimen. At step 62, tumor tissue samples from the patient are collected. The samples may be collected via biopsy or surgery, for example. Next, at step 64, transcriptome profiling (RNA detection and quantification) of the samples collected at step 62 is performed. Any suitable technology/platform designed to profile the transcriptome of samples, e.g., gene expression arrays or next-generation sequencing, can be used at step 64.

At step 66, expression quantification of the genes involved in oncogenesis identified in Stage I of the process shown and described in connection with FIG. 1 (see step 14 of FIG. 1) are extracted. Data derived using different platforms can be transformed. Eventually, at step 68, the transcriptome of the tumor cells can be mapped to the reduced-dimension metagene space. For example, as described above, the metagene space can be a 15-metagene space.

Using the metagene representation of the tumor as input for the COLOXIS model, at step 70, the model computes the probability or a binary call to indicate whether the tumor cells from the patient will respond to FOLFOX, oxaliplatin, and/or bevacizumab. A clinician(s) at step 72 can use the prediction by the COLOXIS model 12 to make a treatment decision for the patient.

FIG. 7 is a diagram of a computer system 100 that could be used to implement the embodiments described above. The illustrated computer system 100 includes one or more processors 102 and one or more memory units 104 that are in communication via a data bus and/or electronic data network. For simplicity, only one processor 102 and one memory unit 104 are shown in FIG. 7. The memory 104 may store various software modules 106, 108, 110, and 112 that comprise software or computer instructions to be executed by the processor 102. For example, the TCI module 106 may include software for performing the TCI analysis of step 14 of FIG. 1; the DEG extraction module 108 may include software for performing the target DEG extraction of step 26 of FIG. 1; the metagene learning and GSVA module 110 may include software for performing the metagene learning and GSVA at step 30 of FIG. 1; and the machine learning training module 112 may include software for training the model 12 at step 38 of FIG. 1.

The computer system 100 of FIG. 7 could also be used to make patient predictions, according to FIG. 6. The memory 104 may store software for the TCI analysis at step 66; for mapping the transcriptome of the tumor cells to the reduced-dimension metagene space at step 68; and inputting the reduced-dimension metagene space to trained, validated COLOXIS model at step 70 to get the drug response predictions.

The processor(s) 102 may include one or more CPU cores, GPU cores, and/or AI accelerator cores. The memory 104 may comprise primary computer memory, such as a read-only memory (ROM) and/or a random access memory (e.g., RAM). The memory 104 could also comprise secondary memory, such as magnetic or optical disk drives or flash memory, for example. The software modules 106, 108, 110, and 112 may be implemented in computer software using any suitable computer programming language such as .NET, C, C++, or Python, and using conventional, functional, or object-oriented techniques. Programming languages for computer software and other computer-implemented instructions may be translated into machine language by a compiler or an assembler before execution and/or may be translated directly at run time by an interpreter. Examples of assembly languages include ARM, MIPS, and x86; examples of high-level languages include Ada, BASIC, C, C++, C#, Python, R, COBOL, Fortran, Java, Lisp, Pascal, Object Pascal, Haskell, ML; and examples of scripting languages include Bourne shell script, JavaScript, Python, Ruby, Lua, PHP, and Perl. The various data used in the process of FIG. 1 may be stored in primary, secondary, tertiary, and/or offline (e.g., cloud) storage.

Computer system 100 can be of varying types including a workstation, server, computing cluster, blade server, server farm, or any other data processing system or computing device. The computer system 100 could be, for example, a server on a cloud network. Due to the ever-changing nature of computers and networks, the description of computer system 100 depicted in FIG. 7 is intended only as a specific example for the purposes of illustrating some implementations. Many other configurations of computer system 100 are possibly having more or fewer components than the computer system depicted in FIG. 7.

In various embodiments, therefore, the present invention provides novel causal methods for searching genes involved in the disease development of CRCs, which serve as more informative features for detecting heterogeneous responses to drugs. The present invention also provides novel feature construction methods (the discovery of metagenes) to lay a foundation for building stable predictive models. The COLOXIS model can predict the prognosis (outcomes) of patients treated with FULV only. This can be used to predict who has a better outcome if treated with FULV. COLOXIS prediction can also be prognostic of outcomes of patients treated with FOLFOX. This can be used to predict a patient's outcome when treated with FOLFOX.

The COLOXIS model also predicts the benefit of oxaliplatin in treating patients with CRC. This can enable a clinician to determine whether a CRC patient should be treated with oxaliplatin. Accurate decisions will increase treatment efficacy as well as prevent overtreatment with oxaliplatin that only causes adverse effects.

The COLOXIS model is also predictive of the benefit of FOLFOX+Bev in the CRC adjuvant setting. This can enable a clinician to include bevacizumab in adjuvant therapy for a subset of patients to increase the treatment efficacy.

The COLOXIS model can also be used to predict sensitivity to the above regimens (FOLFOX) or single drug (oxaliplatin or bevacizumab) in gastrointestinal (GI) cancers, such as esophageal cancer, gastric cancer, and colorectal cancer, in which the regimen and drugs are commonly used. This will enable clinicians to form individualized regimens by selecting effective drugs to treat GI cancer patients.

In one general aspect, therefore, the present invention is computer-implemented systems and methods for training a machine learning to predict an efficacy of the biological agent for the gastrointestinal cancer, such that an output of the model is usable by a clinician for determining a treatment for a new patient with the gastrointestinal cancer. The method comprises computing, by a computer system 100 that comprises one or more processors 102 that execute instructions stored in computer memory 104, expression values for a set of metagenes from target differentially expressed genes (DEGs) for a gastrointestinal cancer, where the set of metagenes exhibit co-expression patterns with respect to response to a biological agent for patients with the gastrointestinal cancer. The method also comprises training, by the computer system, through machine learning, the model to predict the efficacy of the biological agent for the gastrointestinal cancer, such that an output of the model is usable by a clinician for determining a treatment for a new patient with gastrointestinal cancer.

In various implementations, the method further comprise identifying, by the computer system, identifying the target DEGs for gastrointestinal cancer. This identification can be performed using a tumor-specific causal inference algorithm applied to genomic data and transcriptomic data. The method can further comprise extracting, by the computer system, the set of metagenes by using a clustering analysis to identify a reduced set of metagenes that exhibit clean co-expression patterns and strong drug response signals. The reduced set of metagenes comprises ten to twenty, inclusive, metagenes, and preferably around 15 metagenes. The expression values can be computed using a gene set variation analysis (GSVA).

In various implementations, the model comprises a classifier, such as a logistic regression model.

In various implementations, gastrointestinal cancer comprises a colorectal cancer. The biological agent can comprise, for example, FOLFOX, oxaliplatin, and/or bevacizumab.

In various implementation, GSVA scores for the set of metagenes for a cohort of patients are feature vectors for training the model through machine learning. In such cases, each patient in the cohort can be labeled as a responder or non-responder with respect to the biological agent.

In various implementations, the method further comprises, after training the model: collecting tumor tissue samples of the new patient diagnosed with the gastrointestinal cancer; profiling transcriptome of the samples; mapping the transcriptome to the set of metagenes; and classifying, with the model, the efficacy of the biological agent for the new patient based on the mapping of the transcriptome for the new patient to the set of metagenes.

The examples presented herein are intended to illustrate potential and specific implementations of the present invention. It can be appreciated that the examples are intended primarily for the purposes of illustration of the invention for those skilled in the art. No particular aspect or aspects of the examples are necessarily intended to limit the scope of the present invention. Further, it is to be understood that the figures and descriptions of the present invention have been simplified to illustrate elements that are relevant for a clear understanding of the present invention while eliminating, for purposes of clarity, other elements. While various embodiments have been described herein, it should be apparent that various modifications, alterations, and adaptations to those embodiments may occur to persons skilled in the art with the attainment of at least some advantages. The disclosed embodiments are therefore intended to include all such modifications, alterations, and adaptations without departing from the scope of the embodiments as set forth herein.

What is claimed is:

1. A computer system comprising:
one or more processor cores; and
computer memory in communication with the one or more processor cores, wherein the computer memory stores instructions that when executed by the one or more processor cores cause the one or more processor cores to:
compute expression values for a set of metagenes from target differentially expressed genes (DEGs) for a gastrointestinal cancer, where the set of metagenes exhibit co-expression patterns with respect to response to a biological agent for patients with the gastrointestinal cancer; and
train a model, through machine learning, to predict an efficacy of the biological agent for the gastrointestinal cancer, such that an output of the model is usable by a clinician for determining a treatment for a new patient with the gastrointestinal cancer.

2. The computer system of claim 1, the computer memory stores instructions that when executed by the one or more processor cores cause the one or more processor cores to identify the target DEGs for the gastrointestinal cancer.

3. The computer system of claim 2, the computer memory stores instructions that when executed by the one or more processor cores cause the one or more processor cores to identify the target DEGs for the gastrointestinal cancer using a tumor-specific causal inference algorithm applied to genomic data and transcriptomic data.

4. The computer system of claim 2, the computer memory stores instructions that when executed by the one or more processor cores cause the one or more processor cores to extract the set of metagenes by, using a clustering analysis, identifying a reduced set of metagenes that exhibit clean co-expression patterns and strong drug response signals.

5. The computer system of claim 4, wherein the reduced set of metagenes comprises ten to twenty, inclusive, metagenes.

6. The computer system of claim 4, the computer memory stores instructions that when executed by the one or more processor cores cause the one or more processor cores to compute the expression values using a gene set variation analysis (GSVA).

7. The computer system of claim 1, wherein the model comprises a classifier.

8. The computer system of claim 7, wherein the classifier comprises a logistic regression model.

9. The computer system of claim 1, wherein the gastrointestinal cancer comprises a colorectal cancer.

10. The computer system of claim 9, wherein the biological agent comprises FOLFOX.

11. The computer system of claim 9, wherein the biological agent comprises oxaliplatin.

12. The computer system of claim 9, wherein the biological agent comprises bevacizumab.

13. The computer system of claim 6, wherein GSVA scores for the set of metagenes for a cohort of patients are feature vectors for training the model through machine learning.

14. The computer system of claim 13, wherein each patient in the cohort is labelled as responder or non-responder with respect to the biological agent.

15. A method comprising:
computing, by a computer system that comprises one or more processors, expression values for a set of metagenes from target differentially expressed genes (DEGs) for a gastrointestinal cancer, where the set of metagenes exhibit co-expression patterns with respect to response to a biological agent for patients with the gastrointestinal cancer; and
training, by the computer system, through machine learning, a model to predict an efficacy of the biological agent for the gastrointestinal cancer, such that an output of the model is usable by a clinician for determining a treatment for a new patient with the gastrointestinal cancer.

16. The method of claim 15, further comprising identifying, by the computer system, the target DEGs for the gastrointestinal cancer.

17. The method of claim 16, wherein identifying the target DEGs comprises using a tumor-specific causal inference algorithm applied to genomic data and transcriptomic data.

18. The method of claim 16, further comprising extracting, by the computer system, the set of metagenes by, using a clustering analysis, identifying a reduced set of metagenes that exhibit clean co-expression patterns and strong drug response signals.

19. The method of claim 18, wherein the reduced set of metagenes comprises ten to twenty, inclusive, metagenes.

20. The method of claim 18, wherein computing the expression values comprises using a gene set variation analysis (GSVA).

21. The method of claim 15, wherein the model comprises a classifier.

22. The method of claim 21, wherein the classifier comprises a logistic regression model.

23. The method of claim 22, wherein the gastrointestinal cancer comprises a colorectal cancer.

24. The method of claim 23, wherein the biological agent comprises FOLFOX.

25. The method of claim 23, wherein the biological agent comprises oxaliplatin.

26. The method of claim 23, wherein the biological agent comprises bevacizumab.

27. The method of claim 20, wherein GSVA scores for the set of metagenes for a cohort of patients are feature vectors for training the model through machine learning.

28. The method of claim 27, wherein each patient in the cohort is labelled as responder or non-responder with respect to the biological agent.

29. The method of claim 15, further comprising, after training the model:
collecting tumor tissue samples of the new patient diagnosed with the gastrointestinal cancer;
profiling transcriptome of the samples;
mapping the transcriptome to the set of metagenes; and
classifying, with the model, the efficacy of the biological agent for the new patient based on the mapping of the transcriptome for the new patient to the set of metagenes.
